(19) United States
(12) Patent Application Publication
(10) Pub. No.: US 2019/0048418 A1
(43) Pub. Date: Feb. 14, 2019

(54) SYSTEMS AND METHODS FOR DETERMINING IMPACT OF AGE RELATED CHANGES IN SPERM EPIGENOME ON OFFSPRING PHENOTYPE

(71) Applicant: University of Utah Research Foundation, Salt Lake City, UT (US)

(72) Inventors: Douglas T. Carrell, Salt Lake City, UT (US); Bradley R. Cairns, Salt Lake City, UT (US); Timothy G. Jenkins, Salt Lake City, UT (US); Kenneth I. Aston, Salt Lake City, UT (US)

(21) Appl. No.: 15/912,231

(22) Filed: Mar. 5, 2018

Related U.S. Application Data
(63) Continuation of application No. 14/913,246, filed on Feb. 19, 2016, now abandoned.
(60) Provisional application No. 61/868,540, filed on Aug. 21, 2013.

(51) Int. Cl. C12Q 1/6883; G06F 19/18

(52) U.S. Cl. CPC C12Q 1/6883; C12Q 2600/158; G06F 19/18; C12Q 2600/154; G01N 2800/50

(57) ABSTRACT

Methods, systems, and diagnostic tests, including test kits for assessing an offspring's risk of developing a disease or condition known or suspected to have a causal or contributing relationship to an age related epigenetic event in a paternal germ line are disclosed and described.

### PRIORITY DATA

This application claims the benefit of U.S. Provisional Patent Application Ser. No. 61/868,540, filed Aug. 21, 2013 which is incorporated herein by reference.

### FIELD OF THE INVENTION

The present invention relates to determination of offspring phenotype impact from age related changes in a paternal sperm epigenome. In some aspects, such epigenomic changes may be age associated methylation alterations. Accordingly, the present invention involves the fields of reproductive biology, medicine, and molecular biology.

### DESCRIPTION OF FIGURES

FIG. 1: Shows pyrosequencing results for the LINE-1 global methylation assay. The box plot (A) depicts significantly increased average global methylation with age in paired samples from all 17 donors based on a two tailed t-test (p=0.028; A). Global methylation was also stratified based only on age at the time of collection for each sample from all 17 donors (a total of 34 samples with each donor represented twice). The linear regression graph (B) shows that the analysis confirmed significant increases in global sperm DNA methylation with age (p=0.0062; B).

FIG. 2: Shows graphical representations of the attributes of significant windows identified for both hypermethylation events and hypomethylation events (A and B respectively). These designations are based on UCSC annotation at the regions of interest. Average β-values for all significant windows (hypomethylation and hypermethylation events) for both aged and young (C). Average decrease in β-value for intra-individual hypomethylation events was approximately 3.9% and for hypermethylation events was 3.2%. Also shown are results from the co-localization of nucleosomes testing (every region of known histone retention) as well as histone modifications (H3K4 methylation, and H3K27 methylation) with windows of interest (D). Hypermethylation events were less frequently associated with all retained histones (nucleosomes) and loci with H3K27 methylation when compared to hypomethylation events based on Fisher's Exact Test (p=0.002; p=0.0107). Co-localization of hypermethylation or hypomethylation events with H3K4 methylation was statistically similar.

FIG. 3: Shows chromosomal loci of each altered region. Loci of interest are depicted by the indicator marks. Marks on the right side are hypomethylation events and marks on the left side are hypomethylation events (A). The Correlation Maps app on the USeq platform was used to locate any specific chromosomal enrichment of altered methylation windows (i.e. selected or specified region of chromosomal material). Specifically, the application called any 100 kb region where at least two significantly altered methylation marks were found. All called chromosomal enrichment regions are displayed (B) though none were found to be significantly enriched over the background.

FIG. 4: Shows a graphical representation of the frequency of disease associations within the gene set that was analyzed and compared to the frequency of disease associations for all genes known to be associated with at least a single disease based on GAD annotation. Schizophrenia, bipolar disorder, diabetes mellitus and hypertension were selected as there were at least 3 genes in the small set of identified genes that are associated with these diseases. As shown, bipolar disorder and schizophrenia were more frequently associated with the identified genes than the background set of genes based on Fisher's Exact test with p-values of 0.001 and 0.005 respectively. The frequency of genes associated with hypertension and diabetes mellitus in the two groups was statistically similar.

FIG. 5: Shows graphical representations of various descriptive statistics for both TNXB and DRD4; 2 regions of representative methylation alterations. The alignment track for each gene is displayed in Integrated Genome Browser (IGB) with the associated false discovery rate (FDR) denoting the significance of the change and the absolute log 2 ratio reflecting the magnitude of the alteration (A, B). Scatter plots for each sample from all 17 donors (a total of 34 samples with each donor represented twice) with linear regression lines and associated r2 values were generated (C, D). Regression analysis revealed a significant decrease in methylation with age at both DRD4 and TNXB (p=0.0005 and p=0.003 respectively). Additionally, the average methylation within each window (DRD4 and TNXB) was plotted for each paired sample set and is displayed for each donor (E, F). In all cases but one (donor #2 at DRD4) average methylation decreased at both DRD4 and TNXB with age in each donor.

FIG. 6: Shows a graphical comparison of MiSeq results to the array results mentioned below at 21 representative regions (A). Because beta-values and fraction methylation are generated in a different manner (array vs. sequencing respectively) they are not directly comparable. As such the fractional difference for each loci and each technology was compared. This is accomplished by the following equation: fractional difference=(aged value/young value)−1. (B) The fractional difference between young and aged samples at 15 selected loci as measured by array in the 17 donor samples as well as in the independent cohort (19 samples from individuals >=45 years of age and 47 samples from individuals <25 years of age taken from the general population). On average the fractional difference identified in the independent cohort (as measured by sequencing) was approximately 2.2 times greater in magnitude than was identified in the 17 donors.

FIG. 7 shows a graphical representation of single molecule analysis testing results. These results reveled 3 distinct alterations that occur with age. (A) DRD4 has only slight alterations associated with age because the young cohort (<45) is strongly hypomethylated initially, and aging simply amplifies this. RDMR_2 is representative of many alterations observed in this analysis which had a strong population shift from moderately hypomethylated to hypomethylated. TBKBP1 is representative of sites that had a bimodal distribution methylation patterns in the young group that becomes stabilized with age. (B) In every case (DRD4, RDMR_2, TBKBP1) each region has significant demethylation with age though the magnitude of change varies.

### SUMMARY OF EMBODIMENTS

Aspects of the invention involve the identification and use of numerous genomic regions in sperm that undergo age related changes to DNA methylation. Many of these regions correspond to genes that have been previously implicated in the development of neuropsychiatric disorders including schizophrenia, autism, and bipolar disorder. These disorders have all been shown to occur more frequently in the offspring of older fathers. In addition regions involved in the development of paternal age associated diseases including spinocerebellar ataxia, myotonic dystrophy and Huntington's disease also displayed age related changes to sperm DNA methylation patterns. One increased risk for these diseases in the offspring of older fathers is epigenetic changes to the sperm methylome. The regions identified as well as additional regions may serve as important biomarkers for risk of fathering offspring with these disorders. These biomarkers may be important in men regardless of age because of natural intra-individual variation in the sperm methylome.

Analysis of the sperm DNA methylome as a prognostic tool carries significant advantages. The test is completely noninvasive, requiring only a semen sample, and assessment of the methylation status of male gametes offers the most direct prediction of methylation patterns that can be transmitted to offspring. Such patterns may be predictive of the conditions or diseases recited herein among others.

The data presented herein may serve as a foundation for the sperm diagnostic tests to assess the risk of transmission of epigenetic alterations through the male germ line that may cause disease, or increase the risk of disease development, in offspring. Potential methodologies to screen for important methylation alterations in sperm include without limitation, region specific bisulfate pyrosequencing, array based methylation analysis (e.g. Illumina HurnanMethylation450 array, a custom array, or ethyl DNA immunoprecipitation [MeDIP] array analysis), or methyl sequencing (whole genome, region specific, or methyl capture sequencing, or MeDIP sequencing). Two broad applications include the analysis of risk to patients attempting to conceive, as well as the possible use of selecting sperm using sperm selection procedures that may transmit a lower risk.

In one invention embodiment, a method for identifying a subject at risk for a disease or condition attributable to an age-related epigenetic event in the subject's father is provided. Such a method may include obtaining a sample of the father's sperm; and identifying an age related epigenetic event in the father's sperm methylome that is linked to the disease or condition.

In another invention embodiment, a method for identifying a subject's risk for a disease or condition attributable to an age-related epigenetic event in the subject's father is provided. Such a method may in some aspect include obtaining a sample of the father's sperm; and identifying an age related epigenetic event in the father's sperm methylome that is linked to the disease or condition.

In yet another invention embodiment, a method of assessing a risk for a male subject to contribute to a disease or condition in an offspring to be sired is provided. In some aspects, such a method may include obtaining a sample of the subject's sperm; and identifying an age related epigenetic event in the sperm methylome that is known or suspected to cause or contribute to the disease or condition in the offspring.

In an additional invention embodiment is provided, a method of reducing or eliminating a risk of developing a disease or condition in an offspring which is known to relate to an epigenetic event in a paternal sperm methylome. Such a method can include, for example, identifying a disease or condition of concern; obtaining a sample of the paternal sperm; analyzing the sperm to ascertain the presence or absence of an epigenetic event known to relate to the identified disease or condition; and employing a sperm selection procedure that reduces or eliminates sperm having the identified epigenetic event.

In another invention embodiment, a system is provided for determining an offspring's risk of developing a disease or condition known or suspected to have a causal or contributing relationship (i.e. attributable or attributed) to an age related epigenetic event in a paternal sperm methylome. In one aspect, such a system can include information identifying a disease or condition and correlating the disease or condition with a specific epigenetic event in the paternal sperm methylome; equipment configured to receive a sperm sample from the potential paternal source; equipment configured to analyze the sperm sample and identifying the presence or absence the epigenetic event; and an output that reports analysis results.

A further invention embodiment provides a sperm diagnostic test for assessing a risk of transmitting age related epigenetic alterations through a male germline which are known or suspected to increase a risk of disease or condition development in an offspring. In one aspect, such a test can include information identifying a disease of interest and correlating the disease with a specific epigenetic event in the male's sperm methylome; equipment capable of receiving a sperm sample from the male; and equipment capable of analyzing the sperm sample and identifying the presence or absence the epigenetic event.

An additional invention embodiment provides a diagnostic test kit for facilitating assessment of a risk of transmitting age related epigenetic alterations through a male germline which are known or suspected to increase a risk of disease development in an offspring. In one aspect, such a kit can include information identifying a disease of interest and correlating the disease with a specific epigenetic event in the male's sperm methylome; equipment capable of receiving a sperm sample from the male; and a set of instructions for processing the sperm sample using equipment capable of analyzing the sperm sample and identifying the presence or absence the epigenetic event. In an additional aspect, the set of instructions can information for processing the sperm sample using multiple different techniques and equipment capable of processing the sperm sample and identifying the presence or absence of the epigenetic event.

A number of diseases or conditions can be indicated, or the risk therefore, such as a heightened risk can be indicated by the methods and use of the systems, tests, or kits recited herein. However, in one aspect, the disease or condition can be a mental disease or condition. In another aspect, the mental disease or condition is a member selected from the group consisting of: schizophrenia, autism, and bipolar disorder. In a further aspect, the disease or condition is bipolar disorder and a gene associated with the disorder is a member selected from the group consisting of: BCL11A, ATN1, DRD4, PTPRN2, SSTR5, or a combination thereof. In yet another aspect, the disease or condition is schizophrenia and a gene associated with therewith is a member selected from the group consisting of: CL11A, ATN1, DRD4, PTPRN2, SSTR5, or a combination thereof.

Other diseases or conditions can also be indicated, or the risk therefore, such as a heightened risk or a lowered risk. In one aspect, such diseases or conditions can include without limitation diabetes mellitus, hypertension, spinocerebellar ataxia, myotonic dystrophy, or Huntington's disease as well as others. Nearly any disease or condition known or otherwise correlated with specific epigenetic events in the sperm methylome can be evaluated.

### DESCRIPTION OF EMBODIMENTS

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and “the” include plural referents unless the context clearly dictates otherwise. Thus, for example, reference to “a promoter” includes one or more of such promoters and reference to “the histone” includes reference to one or more of such histones.

In describing and claiming the present invention, the following terminology will be used in accordance with the definitions set forth below.

As used herein, “subject” refers to a mammal of interest that may contribute to or experience a genetic abnormality resulting from an epigenetic abnormality in sperm. Examples of subjects include humans, and may also include other animals such as horses, pigs, cattle, dogs, cats, rabbits, and aquatic mammals.

As used herein, “comprises,” “comprising,” “containing” and “having” and the like can have the meaning ascribed to them in U.S. patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. patent law. “Consisting essentially of” or “consists essentially of” have the meaning generally ascribed to them by U.S. patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Furthermore, it is to be understood that express support is provided herein for exact numerical values even when the term “about” is used in connection therewith.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

The effects of advanced paternal age have only recently become of interest to the scientific community as a whole. This interest has likely arisen as a result of recent studies that suggest an association with increased incidence of diseases and abnormalities in the offspring of older fathers. Specifically, offspring sired by aged fathers have been shown to have increased incidence of neuropsychiatric disorders (autism, bipolar disorder, schizophrenia, etc.), trinucleotide repeat associated diseases (myotonic dystrophy, spinocerebellar atixia, Huntington's disease, etc.), as well as some forms of cancer. Though such reports are interesting, very little is known about the etiology of the increased frequency of diseases in the offspring of older fathers. Among the most likely contributing factors to this phenomenon are epigenetic alterations in the male's (i.e. father's) sperm that can be passed on to the offspring.

These studies are in striking contrast to the previously held dogma that the mature sperm are capable only of the safe delivery of the paternal DNA and little more. However with increased investigation has come mounting evidence that the sperm epigenome is not only well suited to facilitate mature gamete function but is also competent to contribute to events in embryonic development. It has been established that even through the dramatic nuclear protein remodeling that occurs in the developing sperm, involving the replacement of histone proteins with protamines, some nucleosomes are retained. This retention is at important genomic loci for development suggesting that the sperm epigenome is well suited to poise the paternal DNA for embryogenesis. Similar DNA methylation marks in the sperm have been identified as well. Such data support the position that the sperm epigenome is not only well suited to facilitate mature sperm function, but that it also contributes to events beyond fertilization.

The contribution of the sperm appears to reach beyond embryogenesis as well. One study involving the offspring of fathers who were exposed to famine conditions supports the concept that sperm, independent of gene mutation, may be capable of affecting phenotype in the offspring. Recently, studies utilizing animal models have discovered similar patterns that comport with the epidemiological data. Specifically, in male animals fed a low protein diet, offspring have altered cholesterol metabolism in hepatic tissue. One causal candidate that may drive these effects is DNA methylation.

Methylation marks at cytosine residues, typically found at cytosine phosphate guanine dinucleotides (CpGS), in the DNA are capable of regulatory control over gene activation or silencing and are additionally believed to help prevent alternative transcription start sites. These roles are dependent on location relative to gene architecture (promoter, exon, intron, etc.). Because these marks are capable of driving changes that may affect phenotype and are heritable they provide a logical candidate for the inheritance of increased disease susceptibility from the father. Age associated sperm DNA methylation alterations at given loci may in some aspects, contribute to the increased incidence of various diseases that can occur in the offspring of older fathers.

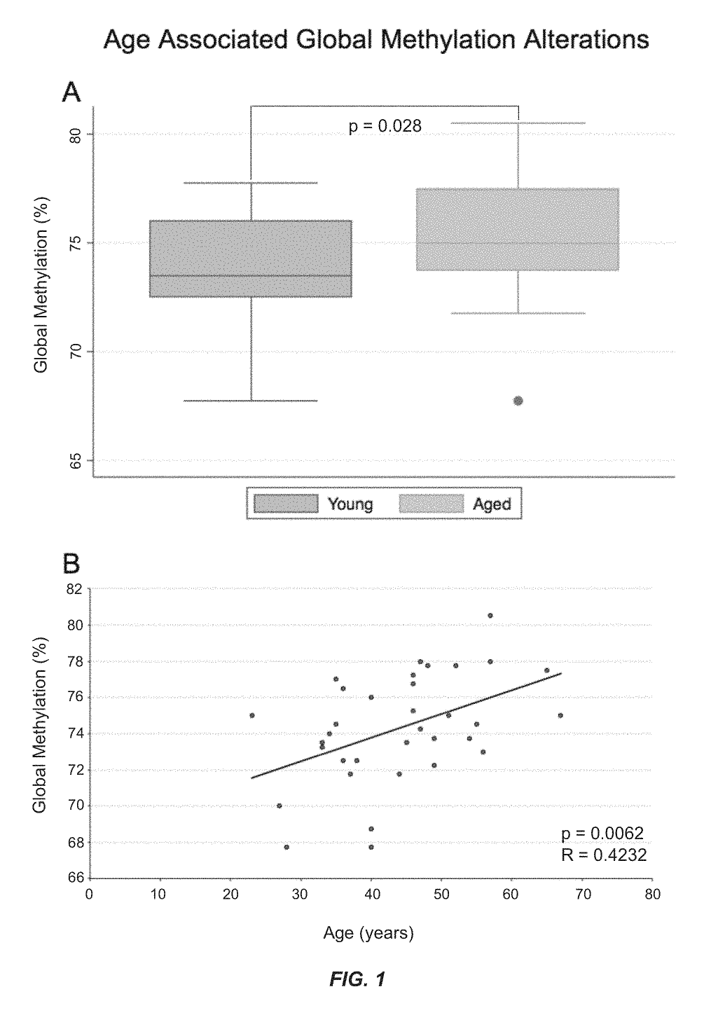

The present inventors have discovered, in general, that sperm DNA methylation marks are robust within individuals as they age, though there are alterations that can occur. Based on pyrosequencing analysis global sperm DNA is significantly hypermethylated with age (FIG. 1). In addition to this global change multiple regions of age-associated methylation alterations were identified. Intra-individual regional methylation alterations between paired samples (young and aged) that consistently occur within the same genomic windows in most or all of the donors screened are also identified. Such alterations occur whether the individual collected the samples in their 20's and 30's or in their 50's and 60's. Specifically, the present window analysis, coupled with regression analysis as an additional filter, reveals a total of 139 regions that are significantly hypomethylated with age (Log 2 ratio ≤−0.2) and 8 regions that are significantly hypermethylated with age (Log 2 ratio ≥0.2) as shown in Table 1. The average called window is approximately 887 base pairs in length and contains an average of 5 CpGs with no fewer than 3 in any significant window. Of the 139 hypomethylated regions 112 are associated with a gene (at either the promoter or the gene body) and of the 8 hypermethylated regions 7 are gene associated. In one case identified 3 significantly hypomethylated windows within a single gene (PTPRN2) were identified. Thus there were a total of 110 genes with age-associated hypomethylation.

TABLE 1
Genomic Features of Significantly Altered Windows

| | | | | | | |
|---|---|---|---|---|---|---|
| ARC | Gene Body | North Shore | N/A | −0.2433 | 65.69 | 0.1902 |
| ATHL1 | Gene Body | Island/South Shore | N/A | −0.2932 | 65.69 | 0.1714 |
| ATN1 | Promoter | North Shelf | N/A | −0.3702 | 65.69 | 0.4421 |
| ATXN7L3 | Promoter | North Shore | N/A | −0.2158 | 65.69 | 0.3413 |
| BEGAIN | Promoter | South Shore | N/A | −0.2747 | 65.69 | 0.4085 |
| BLCAP | Gene Body | North Shore | N/A | −0.2366 | 65.69 | 0.4881 |
| C1orf122 | Promoter | North Shore | N/A | −0.2272 | 65.69 | 0.5157 |
| C6orf48 | Gene Body | South Shore | N/A | −0.2061 | 65.69 | 0.1544 |
| CCDC114 | Promoter | North Shore | N/A | −0.3703 | 65.69 | 0.5512 |
| CCDC144NL | Promoter/Gene Body | Island | N/A | 0.2034 | 65.69 | 0.1989 |
| CFD | Promoter | North Shore | N/A | −0.2795 | 65.69 | 0.3099 |
| CLIC1 | Gene Body | South Shore | N/A | −0.2159 | 65.69 | 0.2098 |
| CNN1 | Promoter/Gene Body | N/A | N/A | −0.2591 | 65.69 | 0.2501 |
| CNTNAP1 | Promoter | North Shore | RDMR | −0.2157 | 65.69 | 0.3904 |
| DLL1 | Gene Body | Island/North Shore | N/A | −0.2937 | 65.69 | 0.1544 |
| DOK1 | Promoter | North Shore | CDMR | −0.2528 | 65.69 | 0.4926 |
| DRD4 | Gene Body | Island | N/A | −0.5705 | 65.69 | 0.3172 |
| EFCAB4A | Gene Body | Island | N/A | −0.3166 | 65.69 | 0.2888 |
| ELANE | Promoter/Gene Body | North Shore | N/A | −0.5163 | 65.69 | 0.1359 |
| GAPDH | Promoter | North shore | RDMR | −0.2191 | 65.69 | 0.2135 |
| GET4 | Promoter | Island/North Shore | N/A | −0.2080 | 65.69 | 0.316 |
| GPANK1 | Gene Body | North Shore | RDMR | −0.2451 | 65.69 | 0.3234 |
| GPR45 | Promoter/Gene Body | Island/North Shore | N/A | −0.2399 | 65.69 | 0.3908 |
| KCNF1 | Gene Body | Island | N/A | −0.3344 | 65.69 | 0.1838 |
| KCNQ1 | Gene Body | Island/North Shore | N/A | −0.2991 | 65.69 | 0.2046 |
| LOC154449 | Promoter | North Shelf | N/A | −0.2064 | 65.69 | 0.122 |
| MIR22HG | Gene Body | North Shore | N/A | −0.2347 | 65.69 | 0.2404 |
| MPPED1 | Gene Body | Island | N/A | −0.2851 | 65.69 | 0.1553 |
| N/A | N/A | HMM Island | N/A | −0.2041 | 65.69 | 0.2629 |
| N/A | N/A | Island/North Shore | N/A | −0.2363 | 65.69 | 0.3355 |
| N/A | N/A | North Shore | N/A | −0.3082 | 65.69 | 0.2066 |
| N/A | N/A | Island/North Shore | N/A | −0.3820 | 65.69 | 0.1795 |
| PCOLCE | Promoter/Gene Body | North Shore | N/A | −0.2438 | 65.69 | 0.1543 |
| PITPNM1 | Promoter | North Shore | N/A | −0.2669 | 65.69 | 0.4935 |
| PPP1R18 | Gene Body | Island/North Shore | N/A | −0.2754 | 65.69 | 0.3867 |
| PRSS22 | Promoter | South Shore | N/A | −0.2486 | 65.69 | 0.5034 |
| PYY2 | Promoter/Gene Body | North Shore | N/A | −0.3241 | 65.69 | 0.6317 |
| SECTM1 | Gene Body | Island | N/A | −0.2568 | 65.69 | 0.3782 |
| SYNE4 | Promoter | North Shore | N/A | −0.2383 | 65.69 | 0.5805 |
| TBKBP1 | Gene Body | Island | N/A | −0.2449 | 65.69 | 0.4863 |
| THBS3 | Promoter/Gene Body | North Shore | N/A | −0.2657 | 65.69 | 0.5953 |
| TNXB | Gene Body | Island | N/A | −0.3319 | 65.69 | 0.2436 |
| UTS2R | Promoter/Gene Body | Island/North Shore | N/A | −0.2767 | 65.69 | 0.2616 |
| ZNF358 | Promoter/Gene Body | Island/North Shore | N/A | −0.2473 | 65.69 | 0.1876 |
| KDM2B | Promoter | South Shore | RDMR | −0.3003 | 65.67 | 0.241 |
| NSG1 | Promoter | North Shore | N/A | −0.2899 | 65.47 | 0.5232 |
| SEZ6 | Gene Body | Island/North Shore | N/A | −0.4530 | 65.05 | 0.43 |
| LMO3 | Promoter | N/A | N/A | −0.3627 | 64.24 | 0.2074 |
| HOXA10 | Promoter/Gene Body | Island/North Shore | N/A | −0.2148 | 64.21 | 0.3354 |
| DAPK3 | Promoter | North Shore | RDMR | −0.3932 | 63.18 | 0.3728 |
| N/A | N/A | Island/North Shore | N/A | −0.3281 | 62.21 | 0.2824 |
| N/A | N/A | South Shore | N/A | −0.2993 | 62.03 | 0.125 |
| NSMF | Gene Body | Island/North Shore | N/A | −0.2249 | 61.30 | 0.329 |
| TOR4A | Promoter | Island/North Shore | N/A | −0.3046 | 61.09 | 0.3998 |
| LDLRAD4 | Promoter | N/A | N/A | −0.2502 | 60.61 | 0.264 |
| N/A | N/A | North Shore | RDMR | −0.2866 | 58.83 | 0.5618 |
| PTPRN2_3 | Gene Body | North Shore | N/A | −0.2391 | 58.31 | 0.151 |
| SSTR5 | Gene Body | Island/North Shore | N/A | −0.2381 | 57.88 | 0.1457 |
| LOC134368 | Gene Body | South Shore | RDMR | −0.2695 | 57.71 | 0.292 |
| GRB7 | Promoter | N/A | N/A | −0.2087 | 57.48 | 0.3144 |
| GNB2 | Gene Body | South Shore | N/A | −0.2238 | 57.45 | 0.1312 |
| SNHG1 | Promoter | North Shore | N/A | −0.2004 | 57.39 | 0.404 |
| LOC653566 | Promoter | N/A | N/A | −0.2929 | 56.31 | 0.2672 |
| N/A | N/A | HMM Island | N/A | −0.2479 | 56.06 | 0.1969 |
| PDE4C | Gene Body | Island/South Shore | N/A | −0.2858 | 55.53 | 0.4673 |
| DLGAP2 | Gene Body | Island/North Shore | N/A | −0.2109 | 55.49 | 0.1296 |
| MRPL36 | Gene Body | North Shore | N/A | −0.2268 | 55.34 | 0.3998 |
| NCOR2 | N/A | HMM Island | N/A | −0.2106 | 55.34 | 0.583 |
| N/A | N/A | North Shore | CDMR | −0.2107 | 54.57 | 0.1157 |
| N/A | N/A | N/A | CDMR | −0.2813 | 52.81 | 0.2763 |
| KCNA7 | Promoter | South Shore | N/A | −0.3664 | 52.24 | 0.5066 |
| CACNA1H | Gene Body | South Shore | N/A | −0.2855 | 51.96 | 0.1695 |
| IRS4 | Gene Body | North Shore | RDMR/CDMR | −0.2273 | 51.23 | 0.2364 |
| KRT19 | Promoter | South Shore | N/A | −0.2701 | 51.08 | 0.3463 |
| LRFN2 | Gene Body | North Shore | RDMR | −0.2525 | 51.08 | 0.2967 |
| WFDC1 | Gene Body | Island | N/A | −0.2966 | 50.49 | 0.2675 |
| APBA2 | Promoter | N/A | N/A | −0.3989 | 50.10 | 0.3216 |
| USP36 | Gene Body | North Shore | RDMR | −0.3108 | 49.92 | 0.2693 |
| PAX2 | Gene Body | South Shore | N/A | −0.3545 | 49.15 | 0.2825 |
| PTPRN2_1 | Gene Body | North Shore | N/A | −0.2828 | 48.41 | 0.3052 |
| N/A | N/A | North Shore | RDMR | −0.2138 | 47.98 | 0.4739 |
| N/A | N/A | HMM Island | N/A | −0.2144 | 47.75 | 0.2672 |
| UNKL | Promoter/Gene Body | Island/North Shore | N/A | −0.2483 | 47.55 | 0.4327 |
| FAM86JP | Promoter | Island/North Shore | N/A | 0.2012 | 47.43 | 0.2884 |
| TTC7B | Promoter | South Shore | N/A | −0.2192 | 47.25 | 0.5194 |
| FAM86C2P | Promoter/Gene Body | Island | N/A | 0.2310 | 46.89 | 0.2156 |
| GRIN1 | Gene Body | Island/North Shore | N/A | −0.3017 | 46.65 | 0.2898 |
| LFNG | Gene Body | South Shore | N/A | −0.3641 | 46.65 | 0.1898 |
| N/A | N/A | HMM Isalnd | N/A | 0.2835 | 46.65 | 0.3944 |
| N/A | N/A | North Shore | RDMR | −0.3885 | 46.65 | 0.5595 |
| SOHLH1 | Promoter/Gene Body | Island/North Shore | N/A | −0.2081 | 46.39 | 0.1542 |
| N/A | N/A | South Shore | RDMR | −0.3423 | 46.34 | 0.1679 |
| N/A | N/A | Island/North Shore | N/A | −0.2100 | 46.34 | 0.3924 |
| SLC22A18AS | Gene Body | South Shore | N/A | −0.2397 | 46.34 | 0.5081 |
| PURA | Promoter | Island/North Shore | N/A | −0.2042 | 46.08 | 0.4237 |
| NFAT5 | Promoter | North Shore | RDMR | −0.2129 | 46.05 | 0.1748 |
| DMPK | Gene Body | Island | N/A | −0.3335 | 46.04 | 0.2442 |
| LOC100133461 | Promoter | North Shelf | N/A | −0.4967 | 46.04 | 0.3899 |
| N/A | N/A | Island/North Shore | CDMR | −0.2369 | 46.04 | 0.4311 |
| N/A | N/A | HMM Island | N/A | −0.3640 | 46.04 | 0.2529 |
| PTPRN2_2 | Gene Body | Island/North Shore | N/A | −0.2666 | 46.04 | 0.1169 |
| PITX1 | Gene Body | North Shore | CDMR | −0.2952 | 45.96 | 0.1888 |
| ARHGEF10 | Gene Body | N/A | N/A | −0.3564 | 45.72 | 0.2585 |
| N/A | N/A | North Shore | N/A | −0.7087 | 45.72 | 0.222 |
| PALM | Gene Body | Island | N/A | −0.2109 | 45.72 | 0.3631 |
| C7orf50 | Gene Body | North Shore | N/A | −0.2133 | 45.54 | 0.1568 |
| SEMA6B | Gene Body | Island/North Shore | CDMR | −0.3163 | 45.39 | 0.3574 |
| FOXK1 | Gene Body | South Shore | RDMR | −0.4457 | 45.27 | 0.4838 |
| FAM86C1 | Promoter/Gene Body | Island | N/A | 0.2260 | 45.18 | 0.1453 |
| ADAMTS8 | Promoter | South Shore | N/A | −0.2193 | 44.74 | 0.5308 |
| N/A | N/A | North Shore | N/A | −0.2771 | 44.67 | 0.2686 |
| EDARADD | Promoter | North Shore | N/A | −0.2506 | 44.52 | 0.3686 |
| FAM86B2 | Promoter | Island | N/A | 0.2238 | 44.48 | 0.2209 |
| AGRN | Promoter | South Shore | N/A | −0.5087 | 44.46 | 0.3049 |
| LEMD2 | Promoter | North Shore | N/A | −0.2055 | 44.46 | 0.414 |
| MTMR8 | Promoter/Gene Body | Island/North Shore | N/A | 0.2070 | 44.27 | 0.3698 |
| MIR9-3 | Promoter | Island/North Shore | N/A | −0.2235 | 44.17 | 0.4838 |
| KRT7 | Promoter | North shore | N/A | −0.2041 | 44.15 | 0.276 |
| NKX2 | Promoter | Island/North Shore | RDMR | −0.3287 | 44.01 | 0.3185 |
| N/A | N/A | North Shore | N/A | −0.2408 | 43.86 | 0.3225 |
| N/A | N/A | North Shore | RDMR | −0.3785 | 43.86 | 0.6517 |
| N/A | N/A | North Shore | RDMR | −0.3876 | 43.56 | 0.3218 |
| USP6NL | Gene Body | Island | N/A | −0.4037 | 43.54 | 0.1384 |
| N/A | Promoter | North Shore | N/A | −0.2067 | 43.22 | 0.3973 |
| N/A | N/A | Island | N/A | −0.2748 | 42.66 | 0.5203 |
| NBLA00301 | Gene Body | North Shore | RDMR | −0.2964 | 42.35 | 0.5779 |
| AJAP1 | Gene Body | South Shore | RDMR | −0.3908 | 42.06 | 0.1215 |
| CRYBA2 | Gene Body | North Shore | N/A | −0.2093 | 42.06 | 0.587 |
| CTF1 | Promoter | South Shore | N/A | −0.2488 | 42.06 | 0.501 |
| FOXF2 | Gene Body | South Shore | RDMR/CDMR | −0.2036 | 41.96 | 0.3976 |
| MAP4K1 | Promoter | North Shore | N/A | −0.2117 | 41.91 | 0.3082 |
| N/A | N/A | HMM Island | N/A | −0.2422 | 41.86 | 0.2107 |
| BCL11A | Gene Body | N/A | N/A | 0.2415 | 41.79 | 0.2955 |
| N/A | N/A | North Shore | RDMR | −0.2307 | 41.76 | 0.529 |
| LONP1 | Gene Body | Island | N/A | −0.2769 | 41.19 | 0.3134 |
| N/A | N/A | HMM Island | N/A | −0.2885 | 41.19 | 0.3396 |
| TBC1D10A | Gene Body | North Shore | N/A | −0.3085 | 41.19 | 0.528 |
| CALCA | Gene Body | North Shore | N/A | −0.2781 | 40.89 | 0.2362 |
| DNMT3B | Gene Body | South Shore | RDMR | −0.3683 | 40.89 | 0.2687 |
| VAX2 | Gene Body | North Shore | RDMR | −0.2485 | 40.89 | 0.3199 |
| ZFPM1 | Gene Body | Island | N/A | −0.2848 | 40.76 | 0.1458 |
| OXLD1 | Gene Body | North Shore | N/A | −0.2737 | 40.60 | 0.3644 |
| FSCN1 | Gene Body | South Shore | RDMR | −0.3639 | 40.31 | 0.3546 |
| FXYD6 | Promoter | South Shore | N/A | −0.3141 | 40.31 | 0.2952 |
| NADK | Promoter | South Shore | RDMR | −0.2196 | 40.31 | 0.3951 |
| PARP12 | Gene Body | North Shore | CDMR | −0.2035 | 40.31 | 0.3821 |
| TBX5 | Promoter/Gene Body | Island/North Shore | N/A | −0.2904 | 40.13 | 0.3641 |

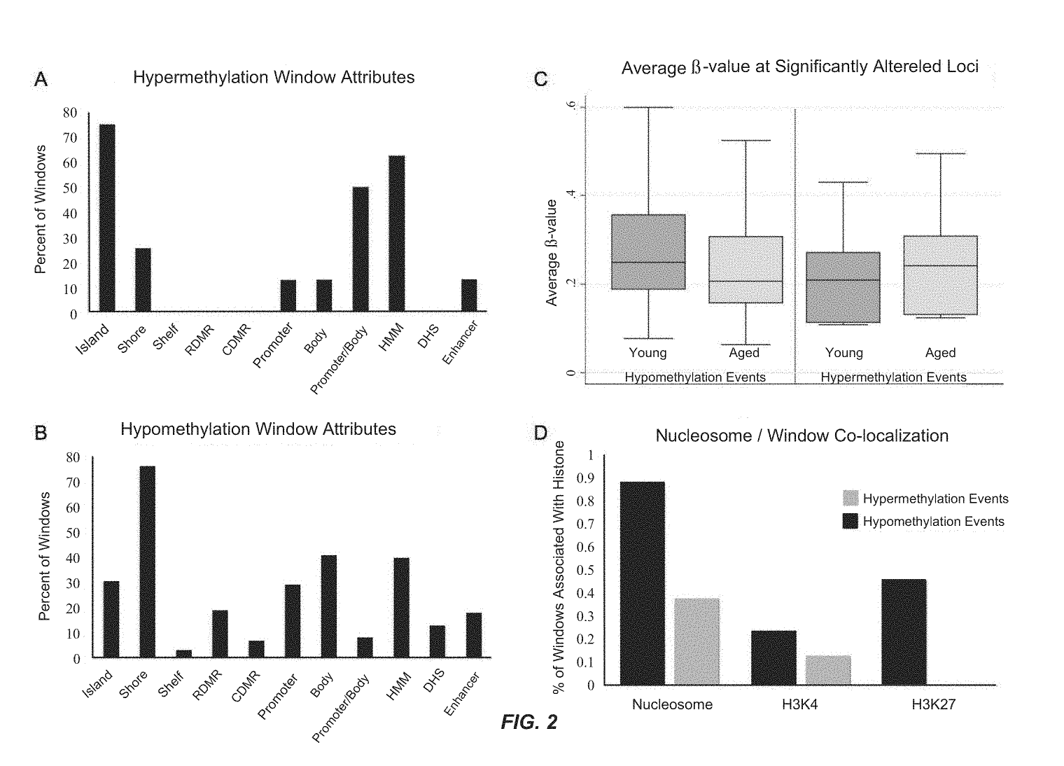

The significant loci identified in the analyses are located at various genomic features. The majority of hypomethylation events with age occur at CpG shores and not in CpG islands themselves, whereas hypermethylation events are more commonly associated with CpG islands as shown in FIG. 2 A-B. In most cases age-associated methylation alterations occur at regions that may likely be of impact to gene transcription (gene body, promoters). However, the data also indicate that these alterations are relatively subtle with intra-individual β-value decreases of approximately 0.039 on average ranging from a β-value decrease of 0.01 to 0.104 between paired samples (young and aged) for hypomethylation events. Similarly, for hypermethylation alterations with age the average β-value increase within a window was approximately 0.032 as shown in FIG. 2C. These alterations all occur in windows with an average initial β-value of <0.6 at the first collection and the majority (68% of Hypomethylation events and 50% of hypermethylation events) are also considered to have intermediate methylation based on conventional standards: β-value <0.2 considered hypomethylated, a value between 0.2 and 0.8 considered intermediate, and a value >0.8 considered hypermethylated.

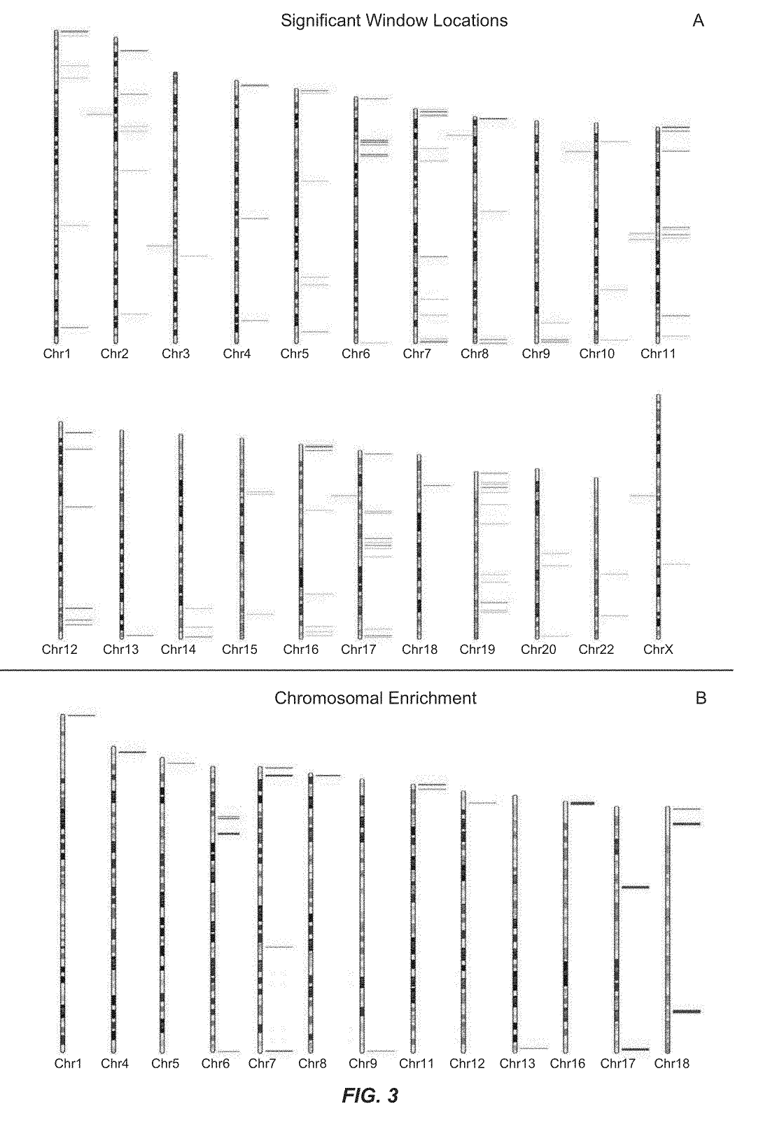

Additionally analyzed is the co-localization of windows of age associated methylation alterations with known regions of nucleosome retention in the mature sperm, as well as regions where specific histone modifications are found based on additional research. It was found that approximately 88% of regions that are hypomethylated with age are found within 1 kb of known nucleosome retention sites in the mature sperm as shown in FIG. 2D. Loci that are hypermethylated with age are far less frequently found in regions of histone retention, with only approximately 37.5% being associated with sites where nucleosomes are found. This difference was significant based on a Fisher's exact test. Similarly, some loci with age-associated hypomethylation are associated with either H3K4 methylation or H3K27 methylation (23% of the loci and 45.3% of the loci respectively). The same co-localization is very rare with hypermethylation events. Additionally analyzed was chromosomal enrichment of these significant marks to determine if there are specific chromosomal regions that are more susceptible to methylation alterations with age. It was found a random distribution of significant age-associated methylation alterations throughout the entire genome with no one chromosomal region being significantly enriched as shown in FIG. 3.

The genes affected by the age associated methylation alterations (those that have alterations that occur at their promoter, or gene body) were analyzed by Pathway, GO and disease association analysis. The results indicate that no one GO term or Pathway is significantly altered in the gene group. Similarly, there were no significant diseases or disease classes associated with the genes identified in this study with the use of the disease association tool on DAVID. However the most significant disease hits (those that were significant prior to multiple comparison correction) have both been suggested to have increased incidence in the offspring of older fathers, namely myotonic dystrophy and schizophrenia.

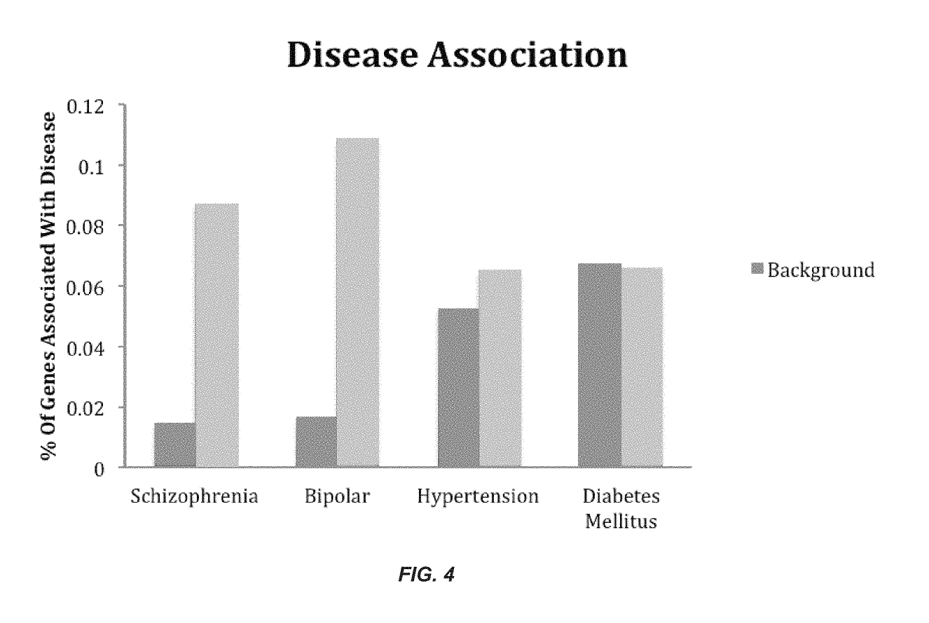

Disease association(s) in the identified genes were searched using the National Institute of Health's (NIH) genetic association database (GAD), which is utilized in DAVID's disease association analysis algorithm. All 117 genes were investigated and were determined to have age associated methylation alterations (110 hypomethylated; 7 hypermethylated) for their various disease associations. A total of 46 genes from the group were confirmed to be associated with either a phenotypic alteration or a disease based on GAD annotation. 4 diseases were identified that had known associations with at least 3 of the genes (diabetes mellitus, hypertension, bipolar disorder and schizophrenia). The frequency of genes associated with these 4 diseases from the identified gene group were analyzed and compared to their frequency in all 11,306 genes known to be associated with either a phenotypic alteration or a disease. This analysis revealed that both bipolar disorder and schizophrenia were more frequently associated with the identified set of genes than the background set of genes based on Fisher's Exact test with p-values of 0.001 and 0.005 respectively as shown in FIG. 4. The frequency of genetic association between the presently identified gene set and the background gene set was statistically similar for both hypertension and diabetes mellitus.

In some aspects, the present invention involves identification of alterations to sperm DNA methylation associated with age. The data reported are in contrast with previous reports of age-associated methylation alterations in somatic cells. For example, some reports suggest age associated global hypomethylation with regional (gene associated) hypermethylation in somatic tissue. In contrast, the present data reveal age-associated hypermethylation globally with a strong bias toward hypomethylation regionally. While the methylation alterations disclosed herein are relatively subtle they are strikingly significant and are common among individuals at various ages and intervals between collections, suggesting that these regions are consistently altered over time in a linear fashion. Importantly, many significantly altered regions are at loci that likely contribute to various diseases known to have increased incidence (i.e. of abnormality or disease) in the offspring of older fathers. Coupling these with the present data demonstrating that no one GO term or Pathway is up or down-regulated in the sperm as a result of the aging process, allows the present inventors to conclude that the alterations observed are a result of regional genomic susceptibility to methylation alteration. This also comports well with the linear nature of the alterations that were observed.

The attributes of regions that were determined to be most susceptible to methylation alterations were analyzed by evaluation of the co-localization of significantly altered loci with regions of known nucleosome retention in the mature sperm. It is discovered that hypomethylation events are most commonly associated with sites of nucleosome retention. This same co-localization was not seen with hypermethylation events.

In some aspects, “selfish spermatogonial selection” may have application in the present invention. This concept states that some gene mutations that are causative of abnormalities in the offspring are beneficial to spermatogenesis and, as a result, are selected for throughout the aging process in the spermatogonial stem cell. Thus, the sperm selfishly select for these mutations at specific loci to the detriment of the offspring. Similarly, the age-associated methylation alterations identified may be in regions that are important to spermatogenesis and thus would be selected for. The hypomethylation events that are selected for could occur as a result of either active or passive demethylation. Specifically, regional transcription activity at loci important in spermatogenesis would likely be accompanied by a relaxed chromatin structure that could result in increased frequency of DNA damage over time. Established methylation marks located within this region could then be passively removed through repair mechanisms in the developing sperm. If the removal of this mark is either beneficial or has no effect on spermatogenesis it will persist, and over time similar marks could accumulate at nearby CpGs ultimately leading to the profiles identified herein. In contrast to this passive methylation removal would be active enzymatic removal of methylation marks in the sperm. In this circumstance hypomethylation in the windows identified is always beneficial to spermatogenesis. In some aspects, the effects identified herein may involve some combination of both mechanisms.

The mechanics of hypermethylation events with age may be an active targeted process with the use of methyltransferase enzymes. However, a possible mechanism for at least a portion of these events can be inferred from the present data. Out of only 7 windows with gene-associated hypermethylation with age, 4 are associated with the FAM86 family of genes that are categorized not by protein function or genomic location but sequence similarity. In some aspects, age associated hypermethylation events at specific loci are driven, either directly or indirectly, by DNA sequence. Interestingly, this family of genes (FAM86) with unknown function has recently been categorized with a larger family of methyltransferase genes. Both active and passive methylation modification can contribute to the herein recited issues.

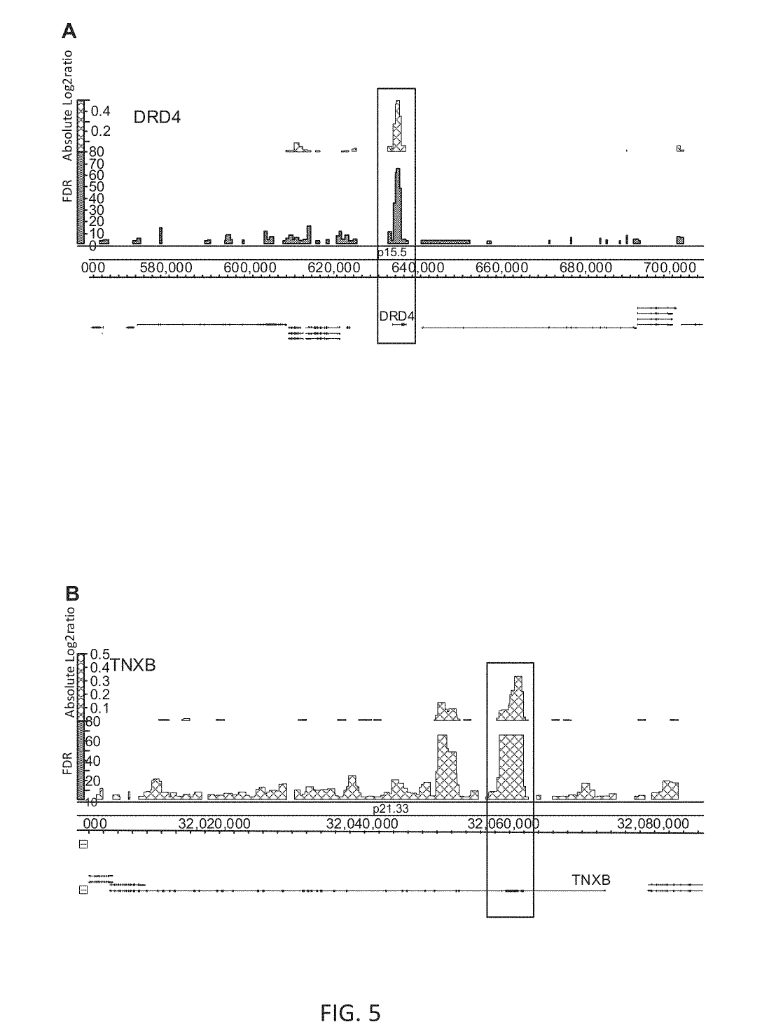

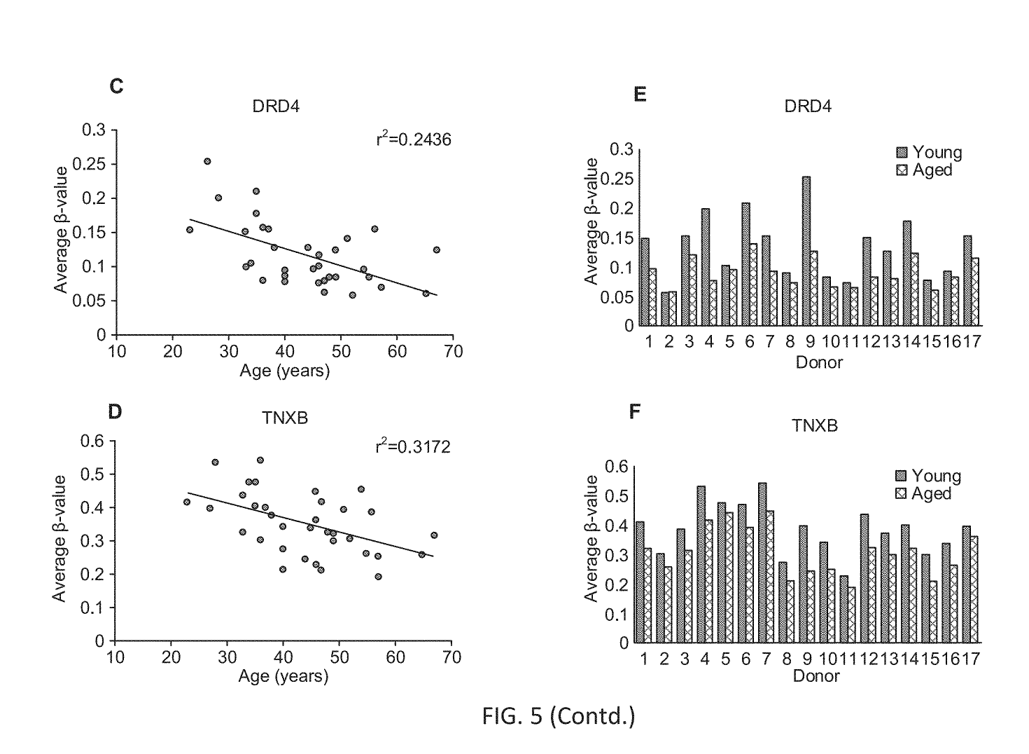

Regardless of the mechanism by which these methylation marks are altered in the sperm over time, it is striking that these changes occur with such consistency between individuals and have such a tight association with age. One limitation of these findings however is the magnitude of alterations discovered. As described earlier the average intra-individual alteration at any given window was approximately a β-value change of 0.039 (effectively a change of 3.9%). Though this seems relatively small, when expanded to include the possible reproductive years in a male (approximately age 20-60) the change would be 10-12%. It is important to understand the nature of what these β-values actually mean in the context of the male gamete. Because of the heterologous nature of the sperm population, a change of this magnitude in average β-value over a window including multiple CpGs can be considered in two different ways. First, that a decrease of 10-12% reflects a complete methylation erasure (from fully methylated to fully demethylated at all CpGs within a given window) in 10-12% of the sperm population. Second, that the observed β-value alterations reflect changes to random CpGs within windows of susceptibility in all sperm, which would manifest in an individual sperm as a hemi-methylated region of interest. The resultant 10-12% change in methylation within every individual sperm (effectively 1 out of every 10 CpGs are demethylated) suggests that every sperm carries similar, yet more subtle, alterations within these windows on average. It is likely that the degree and distribution of these alterations throughout the entire sperm population varies greatly depending on the region of interest and the demethylation process (active or passive). The resultant epigenetic landscape alterations in either case may contribute to disease susceptibility in the offspring despite the small degree of change across the whole population though the increased risk to the offspring may be relatively small. FIG. 5 gives a breakdown of the alterations seen at two representative loci, DRD4 and TNXB.

In some aspects of the present invention the identified age-associated methylation alterations in the mature sperm could be removed through the embryonic demethylation wave. It should be noted that the observed age-associated changes at regions known to be of significance in diseases with increased incidence in the offspring of aged males is striking. The localization of these alterations suggests that the methylation profile in the mature sperm, at specific loci, either contribute to the increased incidence of associated abnormalities in the offspring or that they reflect (are downstream of) changes that are actually causative of the associated abnormalities in the offspring. Moreover, epigenetic alterations are among the most likely candidates to transmit such transgenerational effects, and methylation alterations have been identified that appear capable of contributing to the various pathologies associated with advanced paternal age.

Taken together, these subtle yet highly significant age-associated alterations to the sperm methylation profile are important because of their location and consistency. There are many clear cases in the current set of genes that, if affected, may result in pathologies in the offspring. Dopamine receptor D4 (DRD4) is one of the most influential genes in the pathology of both schizophrenia and bipolar disorder as well as many other neuropsychiatric disorders. The entire DRD4 gene itself is strongly hypomethylated with age as shown in FIG. 5. TNXB may also be associated with schizophrenia. Virtually the entire 1st exon of TNXB is also hypomethylated with age. Additionally, DMPK is associated with myotonic dystrophy, a disease believed to be have paternal age as a risk factor. In fact, DMPK is believed to be the cause of myotonic dystrophy type 1. It is known that this disease is associated with trinucleotide expansion and other data suggests that altered methylation marks are associated with trinucleotide instability. DMPK is known to be altered via trinucleotide repeats. These examples help establish the role that age associated DNA methylation alterations play in the etiology of various diseases associated with advanced paternal age.

Important aspects are two-fold. First, that there are any age-associated alterations common among such a varied study population is remarkable. Specifically, age-associated methylation alterations occur in the sperm regardless of whether the ages between collections are approximately 20 to 30 years of age or 50 to 60 years of age. Second, the increased frequency of genes associated with bipolar disorder and schizophrenia when compared to all genes associated with disease provides a mechanism by which aged fathers may pass on increased susceptibility of these specific disorders known to have increased incidence in the offspring of older fathers. Though frequently hypothesized, this work comprises, to the best of the inventors' knowledge, the first direct evidence suggesting the plausibility of epigenetic alterations in the sperm of aged fathers influencing, or even causing, disease in the offspring.

### Examples

Sample Collection

Samples from 17 sperm donors were accessed (of known fertility) that were collected in the 1990's. These samples were compared to a second group of paired samples from each donor that were collected in 2008. These samples are referred to as young (1990's collection) and aged (2008 collection) samples. The age difference between each collection varied between 9 and 19 years, and the age at first collection (“young” sample) was between 23 and 56 years of age. At every collection donors were required to strictly follow the collection instructions, which include abstinence time of between 2 and 5 days prior to sampling. The whole ejaculate (no sperm selection method was employed) collected at each visit was frozen in a 1:1 ratio with Test Yolk Buffer (TYB; Irvine Scientific, Irvine, Calif.) and stored in liquid nitrogen prior to DNA isolation. Samples were thawed and the DNA was extracted simultaneously to decrease batch effects. Prior to DNA extraction samples underwent somatic cell lysis by incubation in a somatic cell lysis buffer (0.1% SDS, 0.5% Triton X-100 in DEPC H2O) for 20 min on ice to eliminate white blood cell contamination. Samples were visually inspected following lysis to ensure the absence of all potentially contaminating cells before proceeding. Following somatic cell lysis sperm DNA was extracted with the use of a sperm-specific extraction protocol. Briefly, sperm DNA was isolated by enzymatic and detergent-based lysis followed by treatment with RNase and finally DNA precipitation using isopropanol and salt, with subsequent DNA cleanup using ethanol.

Microarray Analysis

Each of the paired samples for the 17 donors (a total of 34 samples) was subjected to array analysis of methylation alterations with age using the Infinium HumanMethylation 450 Bead Chip microarray (Illumina, San Diego Calif.). Extracted sperm DNA was bisulfite converted with EZ-96 DNA Methylation-Gold kit (Zymo Research, Irvine Calif.) according to manufacturer's recommendations. Converted DNA was then hybridized to the array and analyzed according to Illumina protocols at the University of Utah genomics core facility. Once scanned and analyzed for quantities of methylation, or lack of methylation, at each CpG a ß-value was generated by applying the average methylated and unmethylated intensities at each CpG using the calculation: ß-value=methylated/(methylated+unmethylated). The resultant ß-value ranges from 0 to 1 and indicates the relative levels of methylation at each CpG with highly methylated sites scoring close to 1 and unmethylated sites scoring close to 0.

Basic descriptive analyses of the microarray data were performed using Partek (St. Louis Mo.). More in depth analysis was performed using the USeq platform with the application Methylation Array Scanner which identifies regions of altered methylation that are common among individuals with a sliding window analysis. Briefly, paired data from each donor (young and aged) was subjected to a 1000 base pair sliding window analysis where regions of altered methylation with age that are common among donors were called by Wilcoxon Signed Rank Test. To prevent the influence of outliers in the data set methylation for a specific window was reported as a pseudo-median and differences between the young and aged sample are reported as log 2 ratios. Two thresholds were applied to identify windows with significant differential methylation. A Benjamini Hochberg corrected Wilcoxon Signed Rank Test FDR of >=0.0004 and an absolute log 2 ratio >=0.2. To confirm the significance of each of the called windows we subjected the mean β-value within the window for each donor (young and aged samples) to a paired t-test. Following this initial filter each significant window was subjected to a regression analysis to determine the relationship between age and mean methylation within each window. Regression analysis and paired t-tests were performed using STATA 11 software package.

Sequencing Analysis

Each sample was additionally subjected to targeted methylation sequencing at loci determined to be of interest based on microarray analysis. This step was designed to confirm the array results and to provide greater depth of coverage of the CpGs in the windows of interest. Primers for 29 loci were designed using MethPrimer (Li Lab, UCSF). PCR was performed on samples following sperm DNA bisulfite conversion with EZ-96 DNA Methylation-Gold kit (Zymo Research, Irvine Calif.). PCR products were purified with QIA quick PCR Purification Kit (Qiagen, Valencia Calif.) and were pooled for each sample. The Pooled products were delivered to the Microarray and Genomic Analysis core facility at the University of Utah for library prep which included shearing of the DNA with a Covaris sonicator to generate products of approximately 300 base pairs, in preparation for 150 bp paired end sequencing, and the attachment of barcodes for all 34 samples. Multiplex sequencing was then performed on a single lane on the MiSeq platform (Illumina, San Diego Calif.).

Pyrosequencing Analysis

Each sample was subjected to pyrosequencing analysis of a portion of the long interspersed elements (LINE)-1 repeatable element for the purpose of confirming previously determined global methylation changes with age. Briefly isolated sperm DNA samples were submitted to EpigenDx (Hopkinton, Mass.) for the pyrosequencing analysis. Quiagen's PyroMark LINE1 kit was used to determine methylation status at each CpG investigated with the assay. The experiment was performed based on manufacturer recommendations.

GO Term/Pathway/Disease Association Analysis

GO term Analysis was performed with Gene Ontology Enrichment Analysis and Visualization Tool (GOrilla; cbl-gorilla.cs.technion.ac.il). Pathway and disease association analysis was performed on the Database of Annotation, Visualization, and Integrated Discovery (DAVID; david.abcc.ncifcrf.gov) v6.7. Additional disease association analysis was performed directly on the National Institute of Health's Genetic Association Database (GAD; geneticassociationdb.nih.gov).

Additional Statistical Analyses

Fishers exact test was used to determine the differences in frequencies of genes associated with particular diseases between the significant gene group and a background group. This analysis was also used to detect the differences in frequencies of windows that were found in regions of histone retention in the hypomethylation group and the hypermethylation group. Additionally, regression analysis was utilized to determine relationships between age and methylation status at various loci. STATA software package was used to test for significance with these tests (p<0.05).

Independent Cohort Confirmation

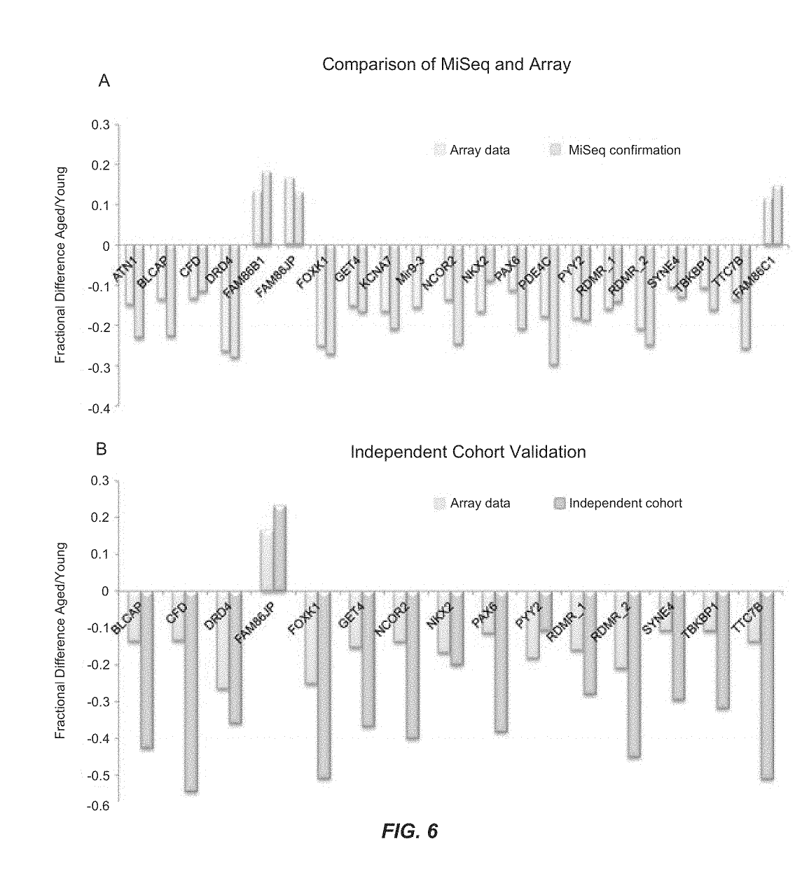

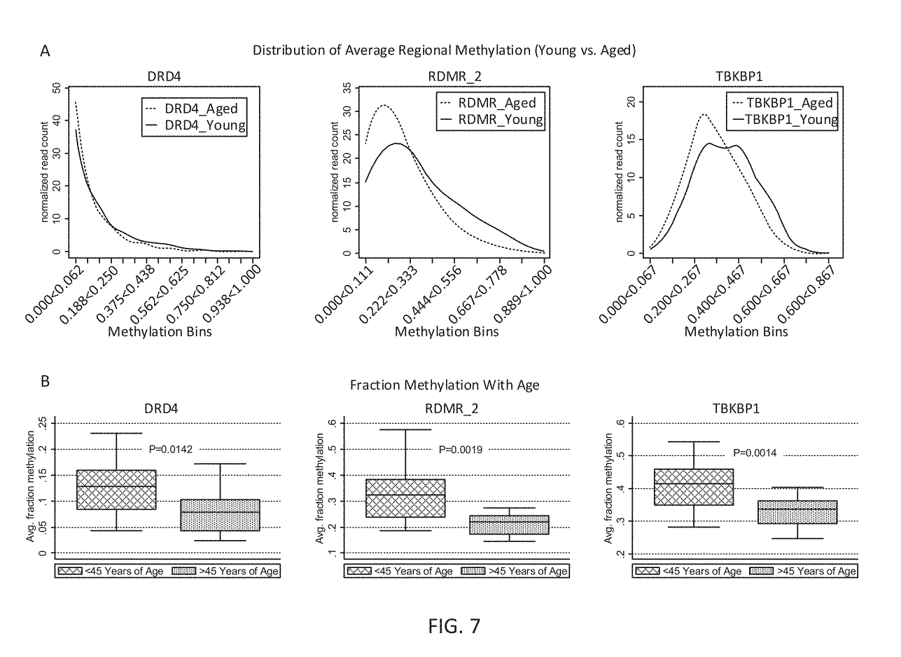

Referring to FIG. 6 is shown a comparison of MiSeq results to the above-recited array results at 21 representative regions (A). This independent cohort testing was performed because beta-values and fraction methylation are generated in different manners (i.e. array vs. sequencing respectively) which prevent a direct comparison. Therefore the fractional difference for each loci and each technology was compared.

The 21 regions were subjected to targeted bisulfite sequencing (on the MiSeq platform) to confirm that the CpGs tiled on the array reflected the entire CpG content within the windows of interest. Specifically, bisulfite converted DNA from each donor (young and aged collections) was amplified via PCR. The PCR was designed to produce amplicons of approximately 300-500 bp that were located within 21 of the regions of significant methylation alteration identified by array. The depth of sequencing was quite robust with an average of 2,252 (SE 371.6) reads per amplicon in each sample. The minimum number of average reads for any one amplicon was 313. In 20 of the 21 gene regions that were analyzed, the array and MiSeq data were similar in both direction and relative magnitude (FIG. 6A). In the one case that did not show a similar trend (hypomethylation with age by array and no change by MiSeq) the amplicon was outside the region of the two CpGs that drove the significance of the window. When comparing the methylation of the approximately 300 bp amplicon to the CpG tiled on the array in that same region only, and not the array CpGs over the entire 1000 bp window, the data are in agreement. Taken together, the sequencing run confirmed that the array data is a good representation of the methylation status at all CpGs in the regions of interest.

To confirm that the sites identified on the array were not only altered in the samples we investigated, but that these loci are also altered with age in the sperm of nonselected individuals in the general population, an analysis was performed on an independent cohort of individuals from two age groups: young, defined as <25 years of age (n=47), and aged, defined as ≥45 years of age (n=19). Average age in the young cohort was 20.46 years of age (SE±0.18), and in the aged cohort 47.71 years of age (SE±0.77). A multiplex sequencing run on sperm DNA from these individuals was performed to probe for 15 different regions of interest that were identified with the array data. Briefly, 15 regions (using bisulfite converted DNA) from each individual (47 young, and 19 aged) were PCR amplified. The PCR was designed to produce amplicons of approximately 300-500 bp that were located within 15 regions of significant methylation alteration identified by array. The depth of sequencing was, again, quite robust with approximately 3,645 (SE±853.4) reads per amplicon in each sample with a minimum average number of reads for any one amplicon of 263. From these data it is confirmed that these genomic regions clearly undergo age-associated methylation alterations (FIG. 6B). Interestingly, the average magnitude of alteration is also much higher in the independent cohort than in the initial paired donor sample study (approximately 2.2 times greater on average). This is particularly remarkable when considering that the average age difference in the independent cohort study was 27.2 years, effectively 2.3 times greater than the average age difference of 12.6 years seen in the paired donor analysis. This further supports our regression data in the paired donor study, which generally suggest a linear relationship of methylation alterations with age at most of the identified genomic loci.

Single Molecule Analysis of Targeted Sequencing

To address the question of the dynamics of sperm population changes associated with the approximately 0.281% change per year identified next generation sequencing data from the paired donor samples was subjected to a novel analysis where the sperm population shifts between the young and aged samples were compared. Because the MiSeq platform produces data for each single nucleotide sequence (each representing the methylation status in a single sperm) it is possible to determine average methylation at each region for all of the amplicons analyzed. 3 general patterns in methylation profile population shifts that resulted in the age-associated methylation alterations were identified. First, regions from subjects were identified whose methylation at an age <45 was strongly hypomethylated, and the methylation profile in individuals >45 years of age is virtually the same, though it is more strongly hypomethylated. In these cases the change is still strikingly significant, but the magnitude of fraction DNA methylation change is minimal. Second, a single population in samples collected at <45 years of age that is shifted toward more hypomethylation in samples collected at >45 years of age can be seen. Last, a bimodal distribution in samples collected <45 years of age that, in samples >45 years of age, is stabilized into a single population was identified. This may indicate at least two sperm subpopulations, which are biased to a single, more hypomethylated sperm population with age. These results could suggest that all of the alterations detected with the array are the result of the entire sperm population being altered in similar subtle ways and not a result of a dramatic alteration in a small portion of the sperm population.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. A method for identifying a subject at risk for a disease or condition attributable to an age-related epigenetic event in the subject's father, comprising:
obtaining a sample of the father's sperm; and
identifying an age-related epigenetic event in the father's sperm methylome that is linked to the disease or condition.

2. (canceled)

3. (canceled)

4. A method of reducing or eliminating a risk of developing a disease or condition in an offspring which is known to relate to an epigenetic event in a paternal sperm methylome, comprising:
identifying a disease or condition of concern;
obtaining a sample of the paternal sperm;
analyzing the sperm to ascertain the presence or absence of an epigenetic event known to relate to the identified disease or condition; and
employing a sperm selection procedure that reduces or eliminates sperm having the identified epigenetic event.

5. The method of claim 1, wherein the disease or condition is a mental disease or condition.

6. The method of claim 5, wherein the mental disease or condition is selected from the group consisting of: schizophrenia, autism, and bipolar disorder.

7. The method of claim 6, wherein the mental disease or condition is bipolar disorder and a gene associated with the disorder is selected from the group consisting of: BCL11A, ATN1, DRD4, PTPRN2, SSTR5, or a combination thereof.

8. The method of claim 6, wherein the mental disease or condition is schizophrenia and a gene associated with therewith is selected from the group consisting of: CL11A, ATN1, DRD4, PTPRN2, SSTR5, or a combination thereof.

9. The method of claim 1, wherein the disease or condition is diabetes mellitus, hypertension, spinocerebellar ataxia, myotonic dystrophy, or Huntington's disease.

10. The method of claim 1, wherein the epigenetic event is either hypomethylation, hypermethylation, or a combination thereof within a selected chromosomal window.

11. A system for determining an offspring's risk of developing a disease or condition known or suspected to have a causal or contributing relationship to an age-related epigenetic event in a paternal sperm methylome comprising:
information identifying a disease or condition and correlating the disease or condition with a specific epigenetic event in the paternal sperm methylome;
equipment configured to receive a sperm sample from a potential paternal source;
equipment configured to analyze the sperm sample and identifying the presence or absence of the specific epigenetic event; and
an output that reports analysis results.

12. The system of claim 11, wherein the disease or condition is a mental disease or condition.

13. The system of claim 12, wherein the mental disease or condition is selected from the group consisting of: schizophrenia, autism, and bipolar disorder.

14. The system of claim 13, wherein the disease or condition is bipolar disorder and a gene associated with the disorder is selected from the group consisting of: BCL11A, ATN1, DRD4, PTPRN2, SSTR5, or a combination thereof.

15. The system of claim 13, wherein the disease or condition is schizophrenia and a gene associated with therewith is selected from the group consisting of: CL11A, ATN1, DRD4, PTPRN2, SSTR5, or a combination thereof.

16. The system of claim 11, wherein the disease or condition is diabetes mellitus, hypertension, spinocerebellar ataxia, myotonic dystrophy, or Huntington's disease.

17. The system of claim 11, wherein the epigenetic event is either hypomethylation, hypermethylation, or a combination thereof within a selected chromosomal window.

18. (canceled)

19. (canceled)

20. (canceled)

21. (canceled)

22. (canceled)

23. (canceled)

24. (canceled)

25. (canceled)

26. (canceled)

27. The method of claim 4, wherein the disease or condition is a mental disease or condition.

28. The method of claim 27, wherein the mental disease or condition is selected from the group consisting of: schizophrenia, autism, and bipolar disorder.

29. The method of claim 28, wherein the mental disease or condition is bipolar disorder and a gene associated with the disorder is selected from the group consisting of: BCL11A, ATN1, DRD4, PTPRN2, SSTR5, or a combination thereof.

30. The method of claim 28, wherein the mental disease or condition is schizophrenia and a gene associated with therewith is selected from the group consisting of: CL11A, ATN1, DRD4, PTPRN2, SSTR5, or a combination thereof.

31. The method of claim 4, wherein the disease or condition is diabetes mellitus, hypertension, spinocerebellar ataxia, myotonic dystrophy, or Huntington's disease.

32. The method of claim 4, wherein the epigenetic event is either hypomethylation, hypermethylation, or a combination thereof within a selected chromosomal window.
